(19) United States
(12) Patent Application Publication
(10) Pub. No.: US 2021/0388496 A1
(43) Pub. Date: Dec. 16, 2021

(54) MOUNTINGS FOR FULLY COATING BODIES, IN PARTICULAR STENTS, BY MEANS OF PECVD

(71) Applicant: BIOTRONIK AG, Bülach (CH)

(72) Inventors: Christian Schwarz, Rostock (DE); Anke Topp, Rostock (DE); Torsten Homuth, Ostseebad Nienhagen (DE)

(21) Appl. No.: 17/290,948

(22) PCT Filed: Nov. 22, 2019
(86) PCT No.: PCT/EP2019/082207

§ 371 Date: May 3, 2021

(30) Foreign Application Priority Data
Nov. 30, 2018 (EP) 18209412.8

(51) Int. Cl. C23C 16/458; A61F 2/82; C23C 16/50; H01J 37/32

(52) U.S. Cl. CPC C23C 16/458; A61F 2/82; C23C 16/50; H01J 37/32715; H01J 2237/3321; A61F 2210/0076; H01J 2237/20214; H01J 2237/2007; A61F 2240/001

(57) ABSTRACT

A device for holding a plurality of workpieces, which each include a circumferential wall structure surrounding an interior, includes a plurality of rotatable holding elements for holding the workpieces. The holding elements are configured to secure the workpieces. The workpieces can be stents and the method of coating can be deposition, particularly plasma enhanced chemical vapor deposition. The holding elements can include at least one connecting element which facilitates electrical charging of the holding elements and hence workpieces when connected to the holding elements by being connected to a charging unit.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP2019/082207, which was filed Nov. 22, 2019, which application claimed priority from European Application EP 18209412, which was filed Nov. 30, 2018.

### FIELD OF THE INVENTION

The invention relates to a device for holding workpieces, in particular stents, in particular when coating the workpieces or stents. Such coatings are used, in particular, to improve the biocompatibility of the particular workpieces/stents. For example, a layer system having at least two layers may be consecutively applied or generated for this purpose. The layer system preferably includes one layer including silicon (Si) and another layer including silicon carbide (SiC), and the layers can be generated by deposition, in particular, by plasma-enhanced chemical vapor deposition (PECVD).

### BACKGROUND

A mounting is known from WO2016/008687A1, in which a respective stent is placed loosely into a rotatable adapter, so that the respective stent is able to rotate along with the rotation of the associated adapter.

### SUMMARY OF THE INVENTION

A device for holding workpieces, in particular in the form of stents, allows the particular workpiece to be fully coated, while ensuring safe manual handling and a high capacity. A preferred device for holding a plurality of workpieces, which each include a circumferential wall structure surrounding an interior of the particular workpiece, includes a plurality of rotatable holding elements, wherein the holding elements are configured to secure (and in particular clamp) the workpieces.

The workpieces can be stents, wherein the wall structure of the respective stent is a lattice structure formed of a plurality of struts.

### BRIEF DESCRIPTION OF THE DRAWINGS

Embodiments and features of the invention shall be described hereafter based on the figures. In the drawings:

FIG. 1 shows perspective views of an embodiment of a device according to the invention, including holding elements formed of wire sections;

FIG. 2 shows a schematic representation of another embodiment of a device according to the invention, including a wave-shaped holding element;

FIG. 3 shows a schematic representation of another embodiment of a device according to the invention, including holding elements made of diverging wire sections;

FIG. 4 shows a perspective view of another embodiment of a device according to the invention, including rotatable shafts for carrying holding elements, which enable a vertical arrangement of the workpieces or stents;

FIG. 5 shows a schematic representation of a holding element of a shaft of the device according to FIG. 4; and

FIG. 6 shows a schematic view of a modification of the device shown in FIGS. 4 and 5, including holding elements made of spring elements.

### DETAILED DESCRIPTION OF THE PREFERRED EMBODIMENTS

A device for holding workpieces, such as stents, includes a plurality of rotatable holding elements, wherein the holding elements are configured to secure (and in particular clamp) the workpieces. The holding elements can include at least one connecting element which facilitates electrical charging of the holding elements and hence workpieces when connected to the holding elements by being connected to a charging unit. The connecting element can be a metal plate being connected to a charger of any kind (charging unit). The device may also include a switch facilitating charging or insulating the holding elements at will of the operator. In one embodiment the charging unit is capable of providing a negative charge to the workpieces.

According to one embodiment of the device, it is further provided that the respective holding element is formed by two parallel wire sections, which are configured to extend through the interior of at least one workpiece in such a way that the at least one workpiece is clamped to the two parallel wire sections.

According to one embodiment, it is provided that the respective wire section has a diameter that is smaller than or equal to 200 μm, and in particular smaller than or equal to 100 μm. According to one embodiment, it is further provided that the respective wire section is made of a metal, in particular of stainless steel, and in particular of the type 1.4301.

According to one embodiment of the device, it is further provided, for clamping the at least one workpiece, that the two wire sections of the respective holding element, based on a state in which no workpiece is clamped to the respective holding element, have a distance perpendicular to a longitudinal axis of the respective wire section that is greater than the inside diameter of the interior of the at least one workpiece to be clamped, wherein the distance is, in particular, at least 0.1 mm larger than said inside diameter.

According to one embodiment of the device, it is further provided that the device is designed to rotate the respective holding element (or the two wire sections) about an associated rotational axis, which extends parallel to the wire sections of the respective holding element and extends, in particular, centrally between the two wire sections of the respective holding element.

According to another embodiment of the device, it is provided that the device is designed to rotate the holding elements (or the respective two wire sections) synchronously about the particular rotational axis.

According to one embodiment of the device, it is further provided that the device includes a carrier for carrying the holding elements, wherein the holding elements are stretched between a first and an opposing second leg of the carrier or extend between the two legs.

According to one embodiment of the device, it is further provided that the respective holding element, so as to rotate about the rotational axis thereof, is coupled to a first gear wheel mounted on the first leg and to a second gear wheel mounted on the second leg.

According to one embodiment of the device, it is further provided that two adjoining first gear wheels of the first leg in each case mesh with one another and/or that two adjoining second gear wheels of the second leg in each case mesh with one another.

According to one embodiment of the device, it is further provided that the first gear wheels are coupled to the second gear wheels via a drive shaft, so that the first and second gear wheels can be rotated synchronously so as to rotate the holding elements about the respective rotational axis, wherein the drive shaft extends parallel to the holding elements.

According to one embodiment of the device, it is further provided that the device includes a drive (fixed to the carrier, for example) for rotating said drive shaft.

According to one embodiment of the device, it is further provided that the respective first gear wheel is arranged on an associated first shaft, which is connected to a first stop cylinder supported on the first leg by way of a spring, wherein the respective spring is configured to pretension the wire sections of a holding element, and wherein the respective first shaft is connected to an associated hook, which includes two curved sections for accommodating a wire connecting section that integrally connects the two wire sections of the particular holding element to one another. The wire sections thus form two mutually connected strands of a wire.

According to one embodiment of the device, it is further provided that the respective hook preferably has a W-shaped design. The device can further include a separate needle, serving as a tool, which is designed to place the wire connecting sections onto the respective hook.

According to one embodiment of the device, it is further provided that the respective second gear wheel is arranged on an associated second shaft, which is connected to a second stop cylinder supported on the second leg, wherein each of the two wire sections of the respective holding element has a free end, wherein the respective second shaft is connected to an associated tensioning cylinder, wherein the respective tensioning cylinder is designed to clamp the two free ends of the wire sections of the particular holding element.

For this purpose, the respective tensioning cylinder can include a notch for each free end for accommodating the respective free end of a wire section, and a respective clamping screw for clamping the respective free end when this is accommodated in the associated notch thereof.

According to one embodiment of the device, it is further provided that the respective holding element is formed by a wire section that is configured to assume a memorized shape for clamping a workpiece when the wire section extends through the interior of the workpiece, wherein the respective wire section is designed to deform, proceeding from an elongated shape, into said memorized shape when a threshold temperature is exceeded.

The wire sections can be separate wire sections, which are formed by separate wires, for example. However, the wire sections can also be integrally joined to one another and form a single wire.

The threshold temperature is preferably above room temperature or above 30°, but preferably below a process temperature present when the workpieces are being coated.

The particular wire section or wire can be brought into the elongated or straight shape, which is maintained by the particular wire section or wire, in particular at room temperature, for threading the workpieces or the stents. This process is reversible. The respective wire section is preferably made of a nickel titanium alloy, and in particular of nitinol.

According to one embodiment of the device, the memorized shape is one of the following shapes: a wave shape, a spiral shape or a zigzag shape.

According to one embodiment of the device, it is further provided that the respective holding element is formed by two diverging wire sections, originating from a wire, which are configured to engage in the wall structure of the workpiece for securing a workpiece.

According to one embodiment of the device, it is further provided that the device includes a plurality of rotatable shafts, which can be rotated by a drive, wherein each shaft carries several of the holding elements.

According to one embodiment of the device, it is further provided that the respective holding element includes a pin that is connected to a shaft and configured to engage in the interior of the workpiece so as to clamp a workpiece, in particular in such a way that the workpiece includes an acute angle with the particular shaft.

According to one embodiment of the device, it is further provided that the multiple holding elements of a shaft are arranged on top of one another, so that multiple workpieces can be clamped along the shaft on top of one another to the particular holding element.

According to one embodiment of the device, it is further provided that a plurality of holding elements, in particular three holding elements, of the holding elements of a shaft project in each case from a holding ring, wherein the respective holding ring surrounds the associated shaft in the circumferential direction. The respective holding ring can have an open annular design, so that the holding ring can be placed onto the particular shaft.

Accordingly, a plurality of (in particular three) workpieces can further be arranged next to one another in the circumferential direction of the respective shaft.

The holding elements or holding rings of a shaft can further be arranged in such a way that, in each case, two adjoining holding elements in the vertical direction or along the shaft are arranged offset from one another in the circumferential direction of the shaft.

According to one embodiment of the device, it is further provided that, in each case, two holding elements are formed by a first and a second spring element, wherein the two spring elements each include a first hook-shaped end section and an opposing second hook-shaped end section, and wherein the two spring elements are each arranged in a through-opening of a shaft, so that the end sections of the spring elements project from the shaft, wherein the first hook-shaped end section of the first spring element is located opposite the first hook-shaped end section of the second spring element along the shaft, and wherein the second hook-shaped end section of the first spring element is located opposite the second hook-y) shaped end section of the second spring element along the shaft.

The first hook-shaped end sections are now preferably configured to engage in the wall structure of the workpiece so as to secure the workpiece on the two spring elements. Similarly, the second hook-shaped end sections are preferably configured to engage in the wall structure of a further workpiece so as to secure the further workpiece on the two spring elements.

According to one embodiment, it is provided that two further holding elements are arranged offset from the two holding elements in the circumferential direction of the shaft, wherein the two further holding elements are formed in each case by two further spring elements arranged in two further through-openings of the shaft, wherein the two further through-openings extend orthogonally to the through-openings of the two other spring elements.

According to one embodiment of the invention, it is further provided that multiple holding elements or the spring elements thereof of a shaft are arranged on top of or next to one another along the shaft.

The above-described holding elements can each be surrounded by a housing of the device in which a plasma can be generated, by the device, so as to apply a coating onto the (in particular, rotating) workpieces or stents by plasma-enhanced chemical vapor deposition. The device can include appropriate electrodes for generating the coating.

According to a further aspect of the invention, a method for coating workpieces, using a device according to the invention, is disclosed, wherein the workpieces are held and rotated by the device and provided with a coating. The method is further provided with an embodiment, wherein the workpiece are charged with a negative potential. In such an embodiment the workpieces could be cleaned e.g. with Ar ions. In another embodiment during coating it is preferable that the workpieces are not charged by a potential and that they are insulated.

According to one embodiment of the method, it is provided that the workpieces are provided with a coating by plasma-enhanced chemical vapor deposition, wherein the coating preferably includes a first layer including silicon (Si), and wherein the coating includes a further, second layer that is applied to the first layer and includes silicon carbide (SiC). The workpieces or stents to which the coating is applied are preferably made of metal.

By the device according to the invention, full coating on all sides is advantageously possible, without changing the contacting of the particular workpiece or stents.

The device according to the invention further enables reliable adhesion of the layer to all areas of the workpiece/stent. Moreover, it is possible to avoid any influence on the base material of the workpiece/stent, such as embrittlement of the material as a result of the inclusion of hydrogen, since the stents are suspended in a conducting manner. The device according to the invention consequently allows a higher throughput/capacity, in particular due to better utilization of the space in the device. Finally, with respect to the wire-based holding elements, the invention has a high automatability potential and overall allows the biocompatibility of the treated workpieces/stents to be efficiently increased.

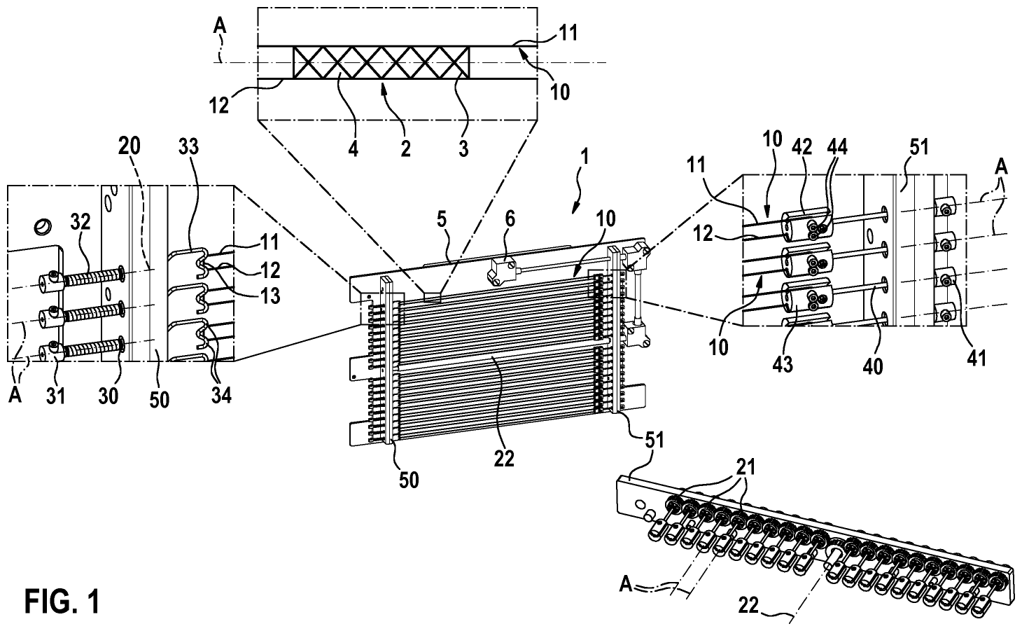

According to FIG. 1, for example, the present invention relates to a device 1 for holding a plurality of workpieces 2, which include a circumferential wall structure 3 surrounding an interior 4 of the respective workpiece 2, wherein the device 1 for holding the workpieces 2 includes a plurality of rotatable holding elements 10, wherein the holding elements 10 are configured to secure or to clamp the workpieces 2, wherein the workpieces 2 are preferably stents 2, wherein the wall structure 3 of the respective stent 2 is, in particular, a lattice structure formed of a plurality of struts and, accordingly, has a plurality of through-openings. Such stents 2 can be cut from a tubular blank by way of laser cutting, for example.

According to the invention, it is further provided, with respect to the method, to use such a device 1 for coating workpieces or stents 2, wherein the coating is carried out, in particular, by PECVD, and the device 1 for holding and, in particular, rotating the workpieces/stents 2 is used in the generation of the coating.

According to the embodiment shown in FIG. 1, it is provided that the respective holding element 10 is formed by two parallel wire sections 11, 12, which are connected in one piece to one another by a wire connecting section 13, and which are configured to extend through the interior 4 of at least one workpiece or stent 2 in such a way that the at least one workpiece 2 is clamped to the two parallel wire sections 11, 12 in that the wire sections 11, 12 push against the inside of the wall structure 3 from the interior 4.

The device according to FIG. 1 is further preferably designed to rotate the respective holding element 10 or the respective two wire sections 11, 12 about an associated rotational axis A, of which several are shown in FIG. 1. The respective rotational axis A extends parallel to the wire sections 11, 12 of the particular holding element 10 associated therewith and extends, in particular, centrally between the two wire sections 11, 12.

Moreover, it is preferably provided that the device includes a drive 6 and a drive shaft 22, which are used to rotate the holding elements 10 or the respective two wire sections 11, 12 synchronously about the particular rotational axis A.

Moreover, the device 1 preferably includes a carrier 5 for carrying the holding elements 10 and, in particular, the drive shaft 22 and the drive 6, wherein the holding elements 10 according to FIG. 1 are stretched between a first and an opposing second leg 50, 51 of the carrier 5.

In particular, it is provided in the process that the respective holding element 10, so as to rotate about the rotational axis A thereof, is coupled to a first gear wheel 20 mounted on the first leg 50 (the first gear wheels are not visible in FIG. 1) and to a second gear wheel 21 mounted on the second leg 51, wherein two neighboring first gear wheels 20 of the first leg 50 and two neighboring second gear wheels 21 of the second leg 51 in each case mesh with one another.

The first gear wheels 20 are now coupled to the second gear wheels 21 via said drive shaft 22, so that the mutually opposing first and second gear wheels 20, 21 are synchronously rotatable so as to rotate the holding elements 10 about the respective rotational axis A.

In detail, it is provided, in particular, in the process that the respective first gear wheel 20 is arranged on an associated first shaft 30, which is connected to a first stop cylinder 31 supported on the first leg 50 by way of a spring 32, wherein the respective spring 32 is configured to pretension the wire sections 11, 12 of the associated holding element 10. The respective first shaft 30 is further connected to an associated hook 33, wherein the respective hook 33 includes two curved sections 34 for accommodating the respective wire connecting section 13 that integrally connects the respective two wire sections 11, 12 of the particular holding element 10 to one another. As is apparent from FIG. 1, the respective hook 33 preferably has a W-shaped design.

Furthermore, it is, in particular, provided that the respective second gear wheel 21 is arranged on an associated second shaft 40, which is connected to a second stop cylinder 41, which is supported on the second leg 51. The two wire sections 11, 12 of the respective holding element 10 further each have a free end, wherein the respective second shaft 40 is connected to an associated tensioning cylinder 42, wherein the respective tensioning cylinder 42 is designed to clamp the two free ends of the wire sections 11, 12 of the associated holding element 10. For this purpose, the respective tensioning cylinder 42 can include a notch 43 for each free end for accommodating the respective free end of a wire section 11, 12, and a respective clamping screw 44 for clamping the respective free end of a wire section 11 or 12 when this is accommodated in the associated notch 43 thereof.

The device 1 thus allows the workpieces 2 to be efficiently threaded onto the holding elements 10 or wire sections 11, 12. Afterwards, the holding elements 10 or the wire connecting sections 13 can be suspended in the hooks 33. The spring elements 32 allow tensioning of the holding elements 10, which also secures the workpieces on the holding elements 10 or wire sections 11, 12.

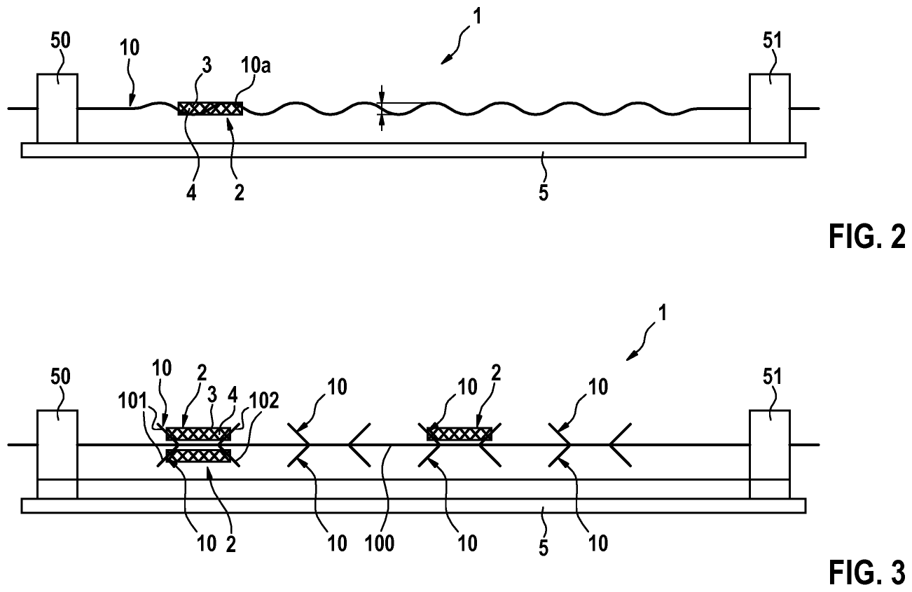

According to an alternative embodiment of the device 1, it is provided according to FIG. 2 that the respective holding element 10 is formed by a wire section 10a that is configured to assume a memorized shape for clamping a workpiece 2 in a reversible manner when the wire section 10a extends through the interior 4 of the workpiece 2, wherein the respective wire section 10a is designed to deform, proceeding from an elongated shape, into said memorized shape when a threshold temperature is exceeded. The threshold temperature is preferably above room temperature or above 30° C., but preferably below a process temperature present when the workpieces are being coated.

The particular wire section 10a can be brought into a stretched or straight shape, which the particular wire section preferably has at room temperature, for threading the workpieces 2 or the stents 2. The respective wire section can be made of a shape memory alloy, for example (such as a nickel titanium alloy, and in particular nitinol). The memorized shape can be a wave shape, for example, according to FIG. 2. Other shapes that allow the workpieces 2 to be clamped are also conceivable. Again, it is possible to thread multiple workpieces 2 consecutively onto a wire section 10a or a holding element 10.

According to a further embodiment of the device 1 according to FIG. 3, it is further provided that the respective holding element 10 is formed by two diverging wire sections 101, 102, originating from a wire 100, which are configured to engage in the wall structure 3 of a workpiece 2 for securing the workpiece 2. Since stents, in general, have a plurality of through-openings, such a design is suitable, in particular, for holding/securing stents 2.

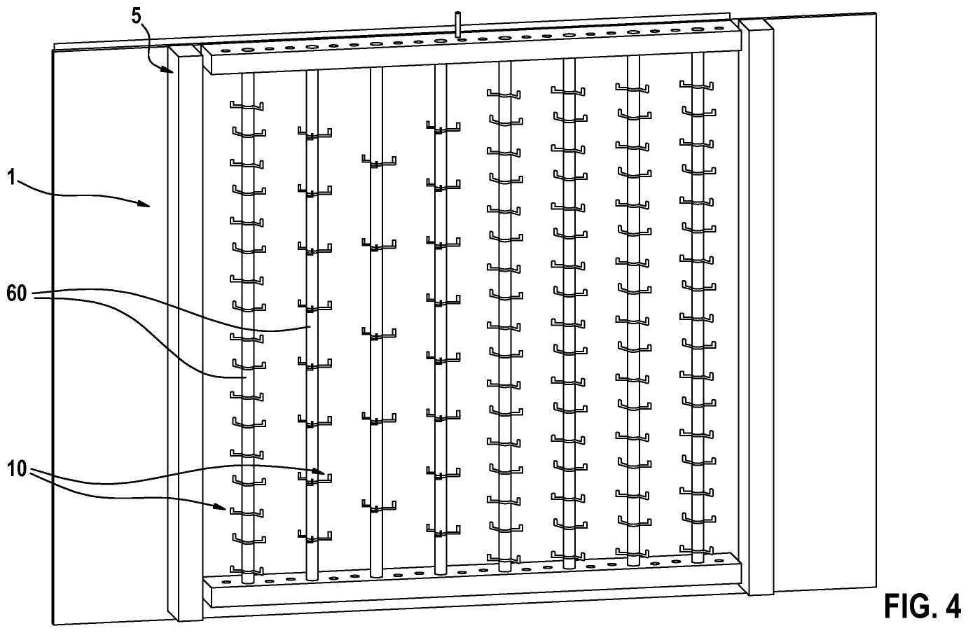

According to the embodiments of the device 1 shown in FIGS. 4 to 6, it is further provided that the device includes a plurality of, in particular, vertically extending and rotatable shafts 60 for rotating the holding elements 10, which can be rotated, for example, by a drive 6 (for example, about a respective vertical rotational axis or about the respective longitudinal axis of the shaft 60), wherein each shaft 60 preferably carries multiple of the holding elements 10.

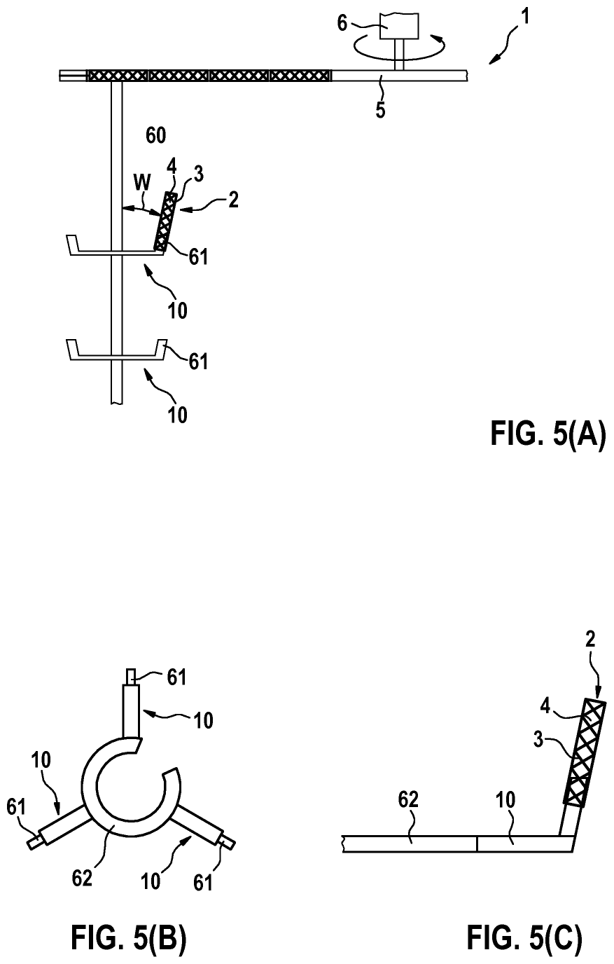

According to the embodiment shown in FIGS. 4 and 5, it is provided that the respective holding element 10 includes a pin 61 connected to a shaft 60 (see, in particular, FIG. 5 (C)), which is configured to engage in the interior 4 of the workpiece 2 so as to clamp a workpiece 2, in particular in such a way that the workpiece 2 includes an acute angle W with the shaft 60 (see FIG. 5 (A)). The respective pin 61 is preferably designed as a flat body so as to keep the surface area of a contact with the inside of the workpiece/stent 2 small.

In particular, it is provided that the multiple holding elements 10 of a shaft 60 are arranged on top of one another (see FIG. 4), so that multiple workpieces 2 can be clamped along the shaft 60 on top of one another on the particular holding element 10.

Furthermore, it is preferably provided that in each case a plurality of holding elements 10, in particular three holding elements 10, of a shaft 60 project from a holding ring 62 (see FIG. 5 (B)), wherein the respective holding ring 62 surrounds the associated shaft 60 in the circumferential direction of the shaft 60. The respective holding ring 62 can have an open annular design, so that the holding ring 62 can be placed onto the particular shaft 60. Accordingly, a plurality of (in particular three) workpieces 2 can further be arranged next to one another in the circumferential direction of the respective shaft 60. The holding elements 10 or holding rings 62 of a shaft 60 can further be arranged in such a way that, in each case, two adjoining holding elements 10 in the vertical direction or along the shaft 60 are arranged offset from one another in the circumferential direction of the shaft 60 (see FIG. 4).

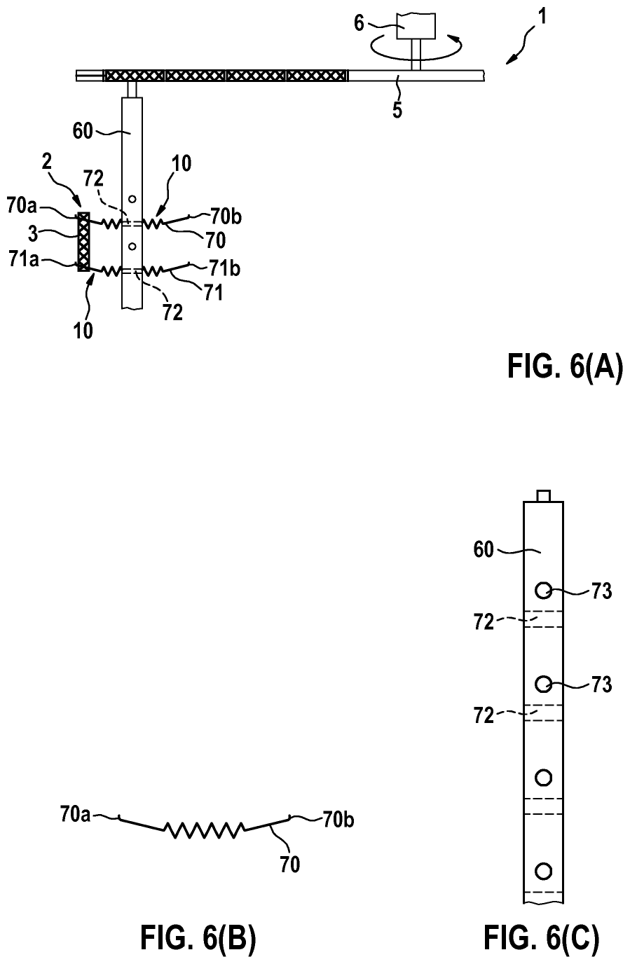

As an alternative, it is further provided according to the embodiment of the device 1 shown in FIG. 6 that, in each case, two holding elements 10 are formed by a first and a second spring element 70, 71, wherein the two spring elements 70, 71 each include a first hook-shaped end section 70a, 71a and an opposing second hook-shaped end section 70b, 71b (see FIGS. 6 (A) and (B)), and wherein the two spring elements 70, 71 are each arranged in a through-opening 72 of a shaft 60, so that the end sections 70a, 70b, 71a, 71b of the spring elements 70, 71 protrude from the shaft 60 or out of the corresponding through-opening 72, wherein the first hook-shaped end section 70a of the first spring element 70 is located opposite the first hook-shaped end section 71a of the second spring element 71, arranged therebeneath, along the shaft 60. Similarly, the second hook-shaped end section 70b of the first spring element 70 is located opposite the second hook-shaped end section 71b of the second spring element 71, arranged therebeneath, along the shaft 60, wherein the first hook-shaped end sections 70a, 71a are configured to engage in the wall structure 3 of a workpiece/stent 2 so as to secure the workpiece or the stent 2 on the two spring elements 70, 71, and wherein the second hook-shaped end sections 70b, 71b are configured to engage in the wall structure 3 of a further workpiece or stent 2 so as to secure the further workpiece/stent 2 on the two spring elements 70, 71.

It is further preferably provided (see, in particular, FIG. 6 (C)) that two further holding elements are arranged offset from the two holding elements 10 or 70, 71 in the circumferential direction of the shaft 60, wherein the two further holding elements are each formed by two further spring elements arranged in two further through-openings 73 of the shaft 60, wherein the two further through-openings 73 extend orthogonally to the through-openings 72 of the two other spring elements 70, 71. It is further possible, of course, for multiple holding elements 10 or 70, 71 of a shaft 60 to be arranged on top of or next to one another along the shaft 60.

The above-described holding elements 10 can each be surrounded by a housing of the device 1 in which a plasma can be generated, by the device 1, so as to apply a coating onto the (in particular, rotating) workpieces 2 or stents 2, for example, by plasma-enhanced chemical vapor deposition.

1. A device for holding a plurality of workpieces, which each comprise a circumferential wall structure surrounding an interior, the device comprising a plurality of rotatable holding elements for holding the workpieces, the holding elements being configured to secure the workpieces.

2. The device according to claim 1, wherein the workpieces comprise a stent, and wherein the wall structure comprise a lattice structure formed of a plurality of struts.

3. The device according to claim 1, wherein the holding elements are formed by two parallel wire sections, which are configured to extend through the interior to clamp a workpiece to the two parallel wire sections.

4. The device according to claim 3, wherein each of the holding elements rotates about a central rotational axis (A) that extends parallel to its two parallel wire sections.

5. The device according to claim 4, comprising a carrier for carrying the holding elements having a plurality of first and second legs with each of the holding elements being stretched between a respective first and an opposing second leg of the carrier.

6. The device according to claim 5, wherein each of the holding elements rotates about the rotational axis (A) and is coupled to a respective first gear wheel mounted on the respective first leg and to a second gear wheel mounted on the respective second leg.

7. The device according to claim 6, wherein each respective first gear wheel is coupled to a respective second gear wheel and driven via a drive shaft such that the respective first gear wheel and second gear wheel can be rotated synchronously to rotate a respective holding element about its respective rotational axis wherein the drive shaft extends parallel to the holding elements.

8. The device according to claim 6, wherein each respective first gear wheel is arranged on a respective first shaft, which is connected to a respective first stop cylinder supported on a respective first leg by a respective spring, the respective spring being configured to pretension the respective wire sections, the respective first shaft being connected to a respective hook, which comprises two curved sections accommodating a wire connecting section that integrally connects the two wire sections to one another.

9. The device according to claim 6, wherein each respective second gear wheel is arranged on a respective second shaft that is connected to a respective second stop cylinder supported on a respective second leg, the two wire sections each having a free end, the respective second shaft being connected to a respective tensioning cylinder, the respective tensioning cylinder being configured to clamp the two free ends of the wire sections.

10. The device according to claim 1, each of the holding elements being formed by a wire section configured to assume a memorized shape for clamping a workpiece when the wire section extends through the interior of the workpiece, the wire section being configured to deform from a stretched shape into the memorized shape when a threshold temperature is exceeded.

11. The device according to claim 1, wherein each of the of the holding elements comprises two diverging wire sections originating from a wire, the diverging wire sections being configured to engage in the wall structure.

12. The device according to claim 1, comprising a plurality of rotatable shafts rotated by a drive for rotating the holding elements, each rotatable shaft carrying a plurality of the holding elements.

13. The device according to claim 12, wherein each of the holding elements comprises a pin that is connected to a rotatable shaft and configured to engage in the interior of a workpiece to clamp the workpiece.

14. The device according to claim 12, wherein two associated holding elements of the plurality of rotatable holding elements are formed by a first and a second spring element each comprising a first hook-shaped end section and an opposing second hook-shaped end section, each of the spring elements being arranged in a through-opening of a shaft, so that the end sections of the spring elements project from the shaft, the first hook-shaped end section of the first end section being located opposite the first hook-shaped end section of the second spring element along the shaft, and the second hook-shaped end section of the first spring element being located opposite the second hook-shaped end section of the second spring element along the shaft, the first hook-shaped end sections being configured to engage in the wall structure to secure a first workpiece on the two spring elements, the two hook-shaped end sections being configured to engage in the wall structure of a second workpiece to secure the second workpiece on the two spring elements.

15. (canceled)
